(19) United States
(12) Patent Application Publication
(10) Pub. No.: US 2011/0213338 A1
(43) Pub. Date: Sep. 1, 2011

(54) Nasogastric tubes

(76) Inventor: Larry Paskar, Chesterfield, MO (US)

(21) Appl. No.: 12/660,489

(22) Filed: Feb. 26, 2010

(51) Int. Cl. A61M 25/00

(52) U.S. Cl. 604/523

(57) ABSTRACT

A nasogastric tube having a proximal end and a distal end portion includes an outer tube and an inner element. The inner element is disposed in the outer tube and is movable rotationally and translationally with respect to the outer tube so that the outer tube and inner element interact to vary the shape of the distal end portion of the nasogastric tube to facilitate the movement of the nasogastric tube from the point of insertion at least to the stomach of the living being on which it is being used. The outer tube is moved with respect to an inner element such that distal end portions of the outer tube and inner element form desired shapes corresponding to the interior anatomy of a living being independent of the actual shape of said anatomy. The distal end portions of the outer tube and inner element are both curved or curvable.

### STATEMENT REGARDING FEDERALLY-SPONSORED RESEARCH/DEVELOPMENT

Not applicable.

### REFERENCE TO MICROFICHE APPENDIX

Not applicable.

### BACKGROUND OF THE INVENTION

This invention relates generally to nasogastric tubes, and more particularly to such devices with improved shaping capabilities. Nasogastric tubes, as used herein, is intended to include, but not be limited to, feeding tubes.

Medical tubes are known for introducing fluids into the stomach or intestines and for removing fluids from same. Many of these are nasogastric tubes, which pass through the nose, the nasopharynx, the oropharynx, and the esophagus into the stomach (and, if desired, beyond). Feeding tubes are common examples. Placement of a feeding tube or other nasogastric or nasoenteric tubes in a living being (i.e., a patient or other animal) can frequently be done without fluoroscopic assistance.

Such placement is not, however, always routine. For example, immediately upon insertion in the nasal passage a bend is required to negotiate around the hard and soft palates. Particularly with weighted tubes (tubes having a weight in the distal end thereof) negotiating that bend can be particularly difficult. In the hypopharynx, the tube can frequently track into the trachea instead of into the esophagus, with consequent great risk to the patient if the misplacement is not detected. Similarly, negotiating non-standard but common passages (such as an hiatus hernia can be extremely difficult with commonly available devices. Placement of the distal end of such a tube in the duodenum, if such placement is desired, can be extremely difficult, as can placement through unexpectedly tortuous structures and bends.

Placement of such tubes can be particularly difficult in older or overweight patients. The possible variations in anatomy in the human body cannot be adequately illustrated in two-dimensional drawings. Passageways that in theory diverge from other passageways at a small angle, may diverge at radically different angles in not just one, but all three dimensions, depending upon the patient. Moreover, these variations in anatomy are unknown (and without expensive and time-consuming tests, unknowable) prior to insertion of the tube. Even with fluoroscopic guidance, it is sometimes difficult to determine the proper shape for traversing an unexpected and unpredictable bend.

As a result, insertion of these tubes frequently involves trial and error—which is time-consuming and frustrating to the patient and the health care professional, resulting in prolonged patient discomfort and excessive exposure to radiation. Because of the difficulty involved in intubating non-standard passageways, sometimes the procedure is unsuccessful.

### SUMMARY OF THE INVENTION

Among the various objects and features of the present invention may be noted the provision of an improved nasogastric tube and method of using same.

A second feature is the provision of an improved nasogastric tube which allows the formation of a shape to be viewed by the user in situ, thereby facilitating the formation of the precise shape(s) needed to access desired passageways and organs.

A third feature is the provision of an improved nasogastric tube with improved ability to pass through tortuous or restricted passages in the body.

Briefly, in a first aspect of the present invention a nasogastric tube having a proximal end and a distal end portion includes an outer tube and an inner element. The outer tube is sized to be inserted in a nasal passage of a living being, and is sufficiently long to extend from the exterior of the living being through the nasal passage to a stomach of the living being. The outer tube is sufficiently flexible to adapt to the shape of passages through which it extends to the stomach. The outer tube has a distal end portion, a proximal end, and a passage extending therethrough. The inner element is disposable in the outer tube and extends therethrough so that a distal end portion of the inner element is at least adjacent the distal end of the outer tube. The inner element is movable rotationally and translationally with respect to the outer tube so that the outer tube and inner element interact to vary the shape of the distal end portion of the nasogastric tube to facilitate the movement of the nasogastric tube from said nasal passage at least to said stomach.

In a second aspect of the present invention, a method of shaping a nasogastric tube having a proximal end and a distal end portion includes the steps of moving an outer tube with respect to an inner element such that distal end portions of the outer tube and inner element form desired shapes corresponding to the interior anatomy of a living being independent of the actual shape of the anatomy. The outer tube is sized to be inserted in a nasal passage of the living being, and is sufficiently long to extend from the exterior of said living being through the nasal passage to a stomach of the living being. The outer tube is sufficiently flexible to adapt to the shape of passages through which it extends to the stomach, and has a distal end portion, a proximal end, and a passage extending therethrough. The inner element is disposed in said outer tube and extends therethrough so that the distal end portion of the inner element is at least adjacent the distal end portion of the outer tube during shaping. The inner element is movable with respect to the outer tube so that the outer tube and inner element interact to vary the shape of the distal end portion of the nasogastric tube (that is, the combined distal end portions of the outer tube and the inner element) as required by the interior anatomy of said living being. Other objects and features will be in part apparent and in part pointed out hereinafter.

### BRIEF DESCRIPTION OF THE DRAWINGS

FIG. 1 is a plan view of a nasogastric tube of the present invention, showing incorrect placement.

FIG. 2 is a view similar to FIG. 1 showing correct placement in the esophagus.

FIG. 3A illustrates improper placement due to an hiatus hernia.

FIG. 3B illustrates possible difficulties in placement into and/or through the stomach.

FIG. 4 illustrates families of up-going shapes for the nasogastric tube.

FIGS. 5 and 6 illustrate families of out-of-plane shapes for the tube of the present invention.

FIG. 7 illustrates families of down-going shapes for the tube of the present invention.

Similar reference characters indicate similar parts throughout the several views of the drawings.

### DESCRIPTION OF THE PREFERRED EMBODIMENT

Referring to the drawings, there is shown a nasogastric tube 11 being improperly inserted into the trachea T of a patient 13. FIG. 2 shows the proper placement in esophagus E. Note that the anatomical structures at the rear of the oral cavity along with the required curved shape to move from the nasal passages to either the trachea or the esophagus at times tend to naturally force a feeding tube or other nasogastric tube toward the trachea.

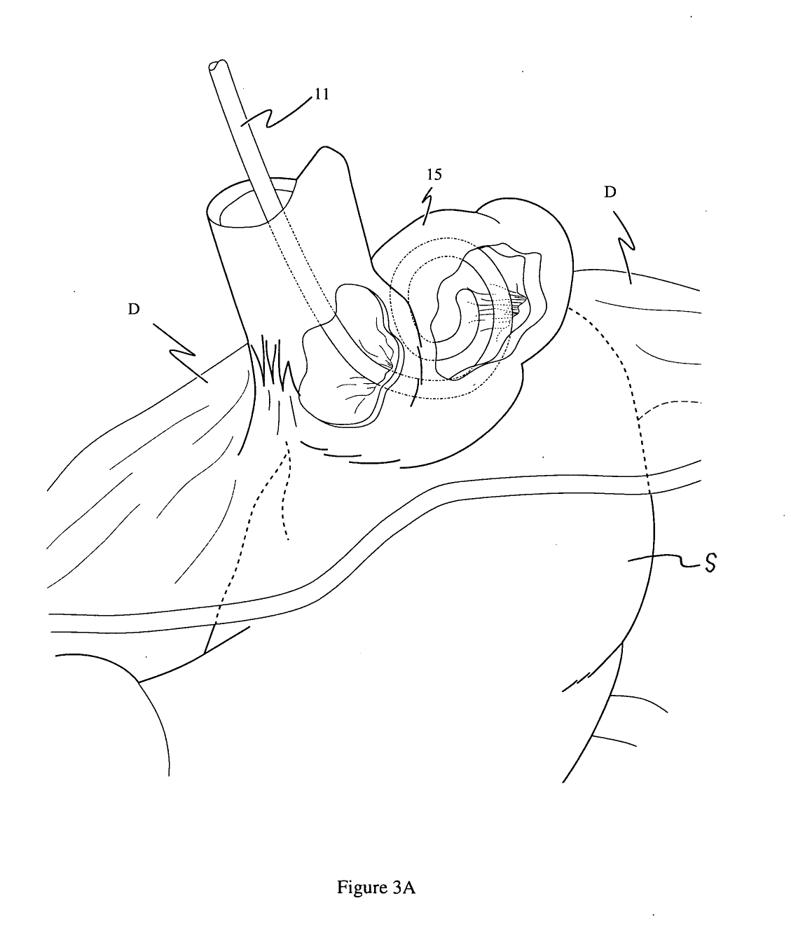

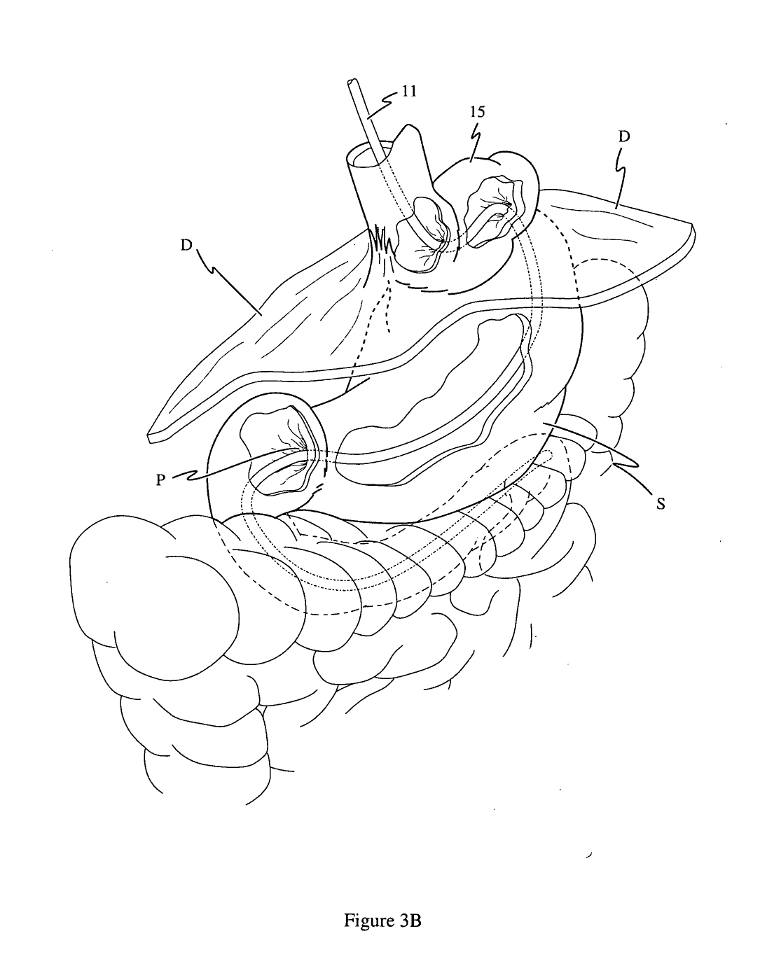

Similar problems may arise lower in the gastro-intestinal tract. For example, in FIGS. 3A and 3B an hiatus hernia 15 is disposed above the diaphragm D and stomach S. Tube 11 can (and in actual practice with conventional tubes does) curl around in the hernia and never reach the desired placement in the stomach. Although the solution to proper movement looks fairly straightforward in FIGS. 3A and 3B, it should be realized that the stomach and the hernia are not necessarily in the same plane. The hernia can be positioned dorsally or ventrally with respect to the stomach and the opening to the stomach can be at a wide variety of angles. Using conventional tubes, this is a serious problem.

Passing through the pyloric valve P into the duodenum is an equally exacting task, even in the best of conditions. Basically, any portion of the gastro-intestinal tract can provide difficult or unexpected situations which make placement of nasogastric tubes difficult. (See FIG. 3B, which illustrates the irregular, non-planar aspects of the problem.)

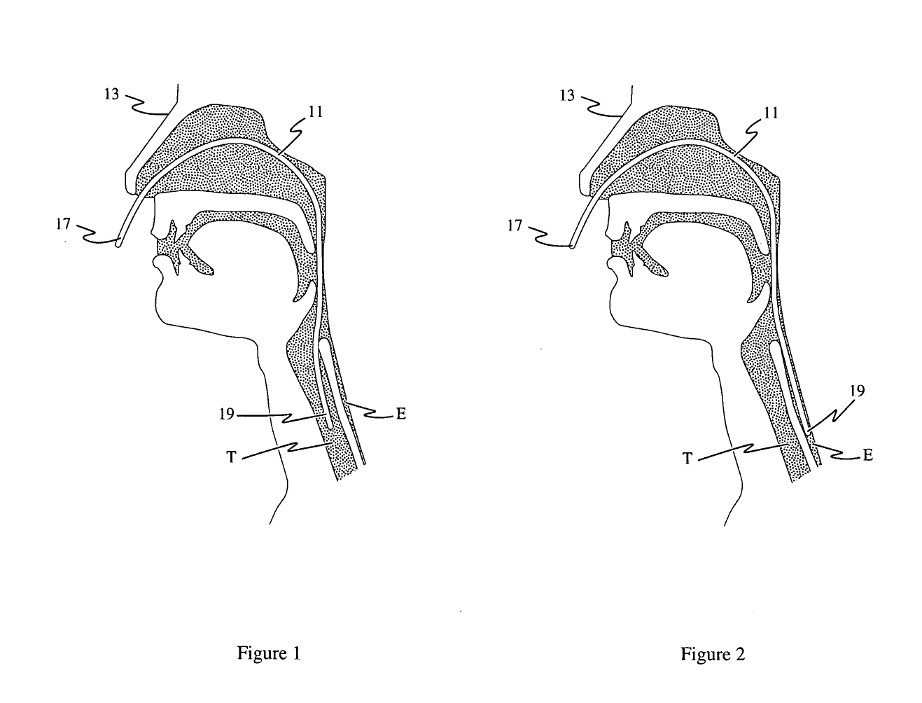

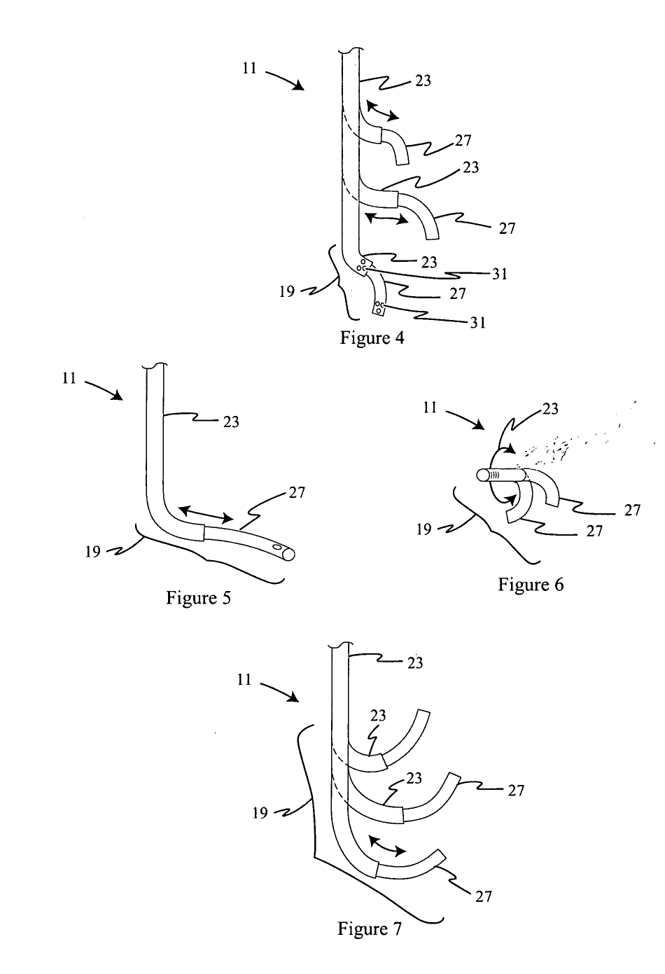

A nasogastric tube 11 of the present invention (FIGS. 4-7) solves these problems. Tube 11 has a proximal end 17 (FIG. 1) and a distal end portion 19. It includes an outer tube 23 sized to be inserted in a nasal passage of a living being 13. The outer tube is sufficiently long to extend from the exterior of the living being through the nasal passage to stomach S. It is sufficiently flexible to adapt to the shape of passages through which it extends to said stomach. The outer tube 23 (like the entire nasogastric tube 11) has a distal end portion, a proximal end, and a passage extending therethrough.

Tube 11 also includes an inner element 27 disposable in outer tube 23 and extending therethrough so that a distal end portion of the inner element is at least adjacent the distal end portion of the outer tube. The inner element is movable rotationally and translationally with respect to the outer tube so that the outer tube and inner element interact to vary the shape of the distal end portion of the nasogastric tube to facilitate the movement of the nasogastric tube from said nasal passage at least to stomach. Translational movement is indicated by the double-headed arrow in FIG. 5, while rotation is best shown in FIG. 6 which shows the inner element rotated to two different positions with respect to outer tube 23. It has been discovered that when the distal end portions of both the outer tube and the inner element are capable of assuming curved shapes a vast multitude of shapes of the composite nasogastric tube 11 can be formed (only a few of which are illustrated in FIGS. 4-7). For example, in FIG. 4, bending outer tube 23 more or less (by a pull-wire or other conventional means) while bending and/or moving the inner element in and out translationally as indicated results in whole families of up-going shapes (an up-going shape being one pointing generally in the direction of insertion of the composite tube 11).

In FIGS. 5 and 6, with the outer tube bent in one plane and rotational movement of the inner curved element, there results whole families of out-of-plane shapes. In addition, in FIG. 7 bending of the outer tube 23 and inner element 27, along with translational movement if desired results in entire families of down-going shapes. (A down-going shape is one pointing generally opposite the direction of insertion of the composite tube 11.) For purposes of this application, the distal ends of the outer tube and the inner element are those curved (or curvable) portions thereof which lie substantially in a plane. When the curves of the outer tube and the inner curved element are at 0 degrees or 180 degrees with respect to each other, the pure up-going and down-going shapes result, while in between those angles various out-of-plane shapes result (with pure out-of-plane shapes at 90 degrees and 270 degrees).

If nasogastric tube 11 is to be used as a feeding tube, it is necessary that either the outer tube 23 or the inner element 27 (or both) have a passage suitable for the passage of nutrients and an opening generally in the distal end portion to allow nutrients to be inserted into stomach S or beyond. In that case, tube 11 is provided with a suitable proximal port for removable attachment to a nutrient source.

It is also preferred, but not required, that nasogastric tube 11 include conventional fluoroscopic imaging markers 31 (see FIG. 4) in the area of the distal end portion of the tube to allow visualization of the shape of the distal end portion as the tube is inserted. Alternatively, the tube 11 itself can be radio-opaque to serve the same purpose.

Although the present invention is particularly well suited for avoiding many of the conventional problems in placing feeding tubes in the stomach, it should be appreciated that the shaping capabilities of this tube make it uniquely suited to solving the difficult problem of placing a tube through the pyloric valve P into the duodenum.

In view of the above it will be seen that the various objects of the invention are achieved and other advantageous results obtained. The examples given herein are for illustrative purposes only and are not to be used in a limiting sense.

What is claimed is:

1. A nasogastric tube having a proximal end and a distal end portion comprising:
an outer tube sized to be inserted in a nasal passage of a living being, said outer tube being sufficiently long to extend from the exterior of said living being through the nasal passage to a stomach of said living being, said outer tube being sufficiently flexible to adapt to the shape of passages through which it extends to said stomach, said outer tube having a distal end portion capable of being curved to define a plane, a proximal end, and a passage extending therethrough;
an inner element disposable in said outer tube and extending therethrough so that a distal end portion of the inner element is at least adjacent the distal end of the outer tube, said distal end portion of the inner element being capable of being curved to define a plane, said inner element being movable rotationally and translationally with respect to the outer tube so that the outer tube and inner element interact to vary the shape of the distal end portion of the nasogastric tube to facilitate the movement of the nasogastric tube from said nasal passage at least to said stomach.

2. The nasogastric tube as set forth in claim 1 wherein the tube has at least one opening at the distal end portion to allow nutrients to be inserted into said stomach.

3. The nasogastric tube as set forth in claim 2 wherein said tube includes a proximal port for removable attachment to a nutrient source.

4. The nasogastric tube as set forth in claim 1 further including fluoroscopic imaging structure to allow visualization of the shape of said distal end portion as said tube is inserted.

5. The nasogastric tube as set forth in claim 1 wherein the distal end portion of the outer tube is capable of assuming a multitude of curved configurations.

6. The nasogastric tube as set forth in claim 1 wherein the distal end portion of the inner element is capable of assuming a multitude of curved configurations.

7. A method of shaping a nasogastric tube having a proximal end and a distal end portion comprising:
moving an outer tube with respect to an inner element such that distal end portions of the outer tube and inner element form desired shapes corresponding to the interior anatomy of a living being independent of the actual shape of said anatomy, said outer tube being sized to be inserted in a nasal passage of the living being, said outer tube being sufficiently long to extend from the exterior of said living being through the nasal passage to a stomach of said living being, said outer tube being sufficiently flexible to adapt to the shape of passages through which it extends to said stomach, said outer tube having a distal end portion, a proximal end, and a passage extending therethrough;
said inner element being disposed in said outer tube and extending therethrough so that the distal end portion of the inner element is at least adjacent the distal end of the outer tube during shaping, said inner element being movable with respect to the outer tube so that the outer tube and inner element interact to vary the shape of the distal end portion of the nasogastric tube as required by the interior anatomy of said living being, said distal end portion of the outer tube being curved or curvable to define a plane and the inner element being curved or curvable to define a plane.

8. The method as set forth in claim 7 wherein shapes formed may be varied as required as the tube is moved along one or more passages in the living being.

9. The method as set forth in claim 7 wherein the shaping occurs under fluoroscopic imaging.

10. The method as set forth in claim 7 wherein the inner element is moved translationally with respect to the outer tube to vary the shape of the nasogastric tube.

11. The method as set forth in claim 7 wherein the inner element is moved rotationally with respect to the outer tube to vary the shape of the nasogastric tube.

12. The method as set forth in claim 7 wherein the inner element is moved rotationally and translationally with respect to the outer tube to vary the shape of the nasogastric tube.

13. The method as set forth in claim 7 wherein the distal end portion of the nasogastric tube assumes a multitude of curved configurations during insertion of said tube.

14. The method as set forth in claim 7 wherein nasogastric tube is formed into a shape inside the living being to avoid placement of said tube in the trachea.

15. The method as set forth in claim 7 wherein the nasogastric tube is formed into a shape inside the living being to negotiate through an hiatus hernia.

16. The method as set forth in claim 7 wherein the nasogastric tube is formed into a shape inside the living being to pass through the stomach into the duodenum.

17. The method as set forth in claim 7 wherein the nasogastric tube is formed into a shape inside the living being to negotiate past the hard and soft palates.
